(19) United States
(12) Patent Application Publication
(10) Pub. No.: US 2012/0238934 A1
(43) Pub. Date: Sep. 20, 2012

(54) COMPRESSION SYSTEM

(75) Inventor: Erik Düring, Sollentuna (SE)

(73) Assignee: St. Jude Medical Systems AB

(21) Appl. No.: 13/512,440

(22) PCT Filed: Nov. 29, 2010
(86) PCT No.: PCT/SE2010/051313

§ 371 Date: May 29, 2012

Related U.S. Application Data
(60) Provisional application No. 61/265,070, filed on Nov. 30, 2009.

(30) Foreign Application Priority Data
Nov. 30, 2009 (SE) 0950917-5

(51) Int. Cl. A61F 13/00

(52) U.S. Cl. 602/53

(57) ABSTRACT

The invention relates to a compression system (1) for compression against a puncture site of a vessel in a patient, comprising: a compression element (4) adapted to apply a pressure against said puncture site; a tightening unit (5) adapted to extend around a part of or the whole of the patient's body to fixate and to tighten the compression element against said puncture site; wherein the compression element is provided with a placement mark (18, 19, 22, 23, 24, 25) indicating where the compression element should be placed in relation to an entrance site through the skin of the patient made to reach said puncture site, in order to apply pressure against said puncture site.

### FIELD OF THE INVENTION

The present invention relates to a compression system for compression of a vessel, and in particular to a compression system which facilitates correct placement of the system.

### BACKGROUND OF THE INVENTION

To access a patient's vascular system for an invasive medical procedure such as catheterization or similar procedures, a puncture is made in e.g. the femoral artery or the radial artery. Following the medical procedure the flow of blood through the puncture site has to be stopped, so that haemostasis may begin as soon as possible. This may be done by using a compression device.

One example of such a compression device is known from WO 2009/000665, describing a femoral compressing device for compressive bearing against the femoral artery of a patient. The device comprises a base plate, an inflatable air cushion, and a manometer connected to the inflatable air cushion. The device is fixed around the patient's body with a belt. In use, the inflatable air cushion is positioned over the femoral artery, and the belt is tightened and secured around the patient's body. To apply pressure to the femoral artery, the inflatable air cushion is inflated by a pump to a certain predetermined pressure, which is read from a pressure gauge.

An example of a compression device for radial artery compression is described in WO 2004/017841. The device comprises a splint with a main body, a first and second strap, a compression strap and a compression pad. The first and second straps are preferably arranged at the longitudinal ends of the splint to fixate a hand, wrist and forearm in the splint. When fixated, the compression strap is used to put pressure on the compression pad, which is to be positioned over the wound or puncture site in the forearm or wrist to occlude blood flow through only the radial artery while allowing blood flow to the hand through the ulnar artery.

It is of major importance that the element that is positioned over the puncture site or wound is positioned correctly, to securely stop the blood flow. In many cases the entrance site for e.g. a catheter through the skin is not located directly above the puncture site in the artery or vein because the catheter often is inserted in an acute angle in relation to the skin. In other words, the entrance site through the skin is offset from the puncture site in the artery or vein. It may therefore be difficult to correctly locate the puncture site in relation to the entrance site.

In the instructions for the FemoStop®, which is a femoral compression device from St. Jude Medical, a user is instructed to place the centre of the dome (e.g. an inflatable air cushion) slightly superior and medial to skin incisions, in order to ensure that the dome is positioned over the actual arterial/venous puncture site. To correctly follow these instructions to position the dome, the user in many cases needs some experience.

The object of the invention is thus to facilitate a correct positioning of a compression device to stop a blood flow from a puncture site.

### SUMMARY OF THE INVENTION

The above-mentioned object is achieved by a compression system for compression against a puncture site of a vessel in a patient according to the independent claim. The compressing system comprises a compression element adapted to apply a pressure against said puncture site, and a tightening unit adapted to extend around a part of, or the whole of, the patients body to fixate and to tighten the compression element against said puncture site. The compression element is further provided with a placement mark indicating where the compression element should be placed in relation to an entrance site through the skin of the patient made to reach said vessel puncture site, in order to apply pressure against the puncture site.

By providing a placement mark on the compression element a user of the system is guided when positioning the compression element, thus facilitating a correct placement of the compression device to stop blood flow in the vessel. The placement mark may be a colour mark or a depression, or a combination of them.

Preferred embodiments are set forth in the dependent claims and in the detailed description.

### SHORT DESCRIPTION OF THE APPENDED DRAWINGS

Below the invention will be described with reference to the description, of which:

FIG. 1 shows an example of a femoral compression device.

FIG. 2 illustrates a compression element positioned over a puncture site of a patient's body.

FIG. 3 schematically illustrates the location of a puncture site in relation to an entrance site for e.g. an introducer.

FIG. 4 illustrates one embodiment according to the invention.

FIG. 5 illustrates another embodiment according to the invention.

FIG. 6 illustrates a further embodiment according to the invention.

FIG. 7 illustrates a still further embodiment according to the invention.

FIG. 8 illustrates a still further embodiment according to the invention.

FIG. 9 illustrates a still further embodiment according to the invention.

FIG. 10 shows an example of a radial compression device.

FIG. 11 illustrates the radial compression device in FIG. 10 in cross-section.

FIG. 12 illustrates one embodiment according to the invention.

FIG. 13 illustrates another embodiment according to the invention.

FIG. 14 illustrates the embodiment in FIG. 12 in cross-section along A-A.

### DETAILED DESCRIPTION OF PREFERRED EMBODIMENTS OF THE INVENTION

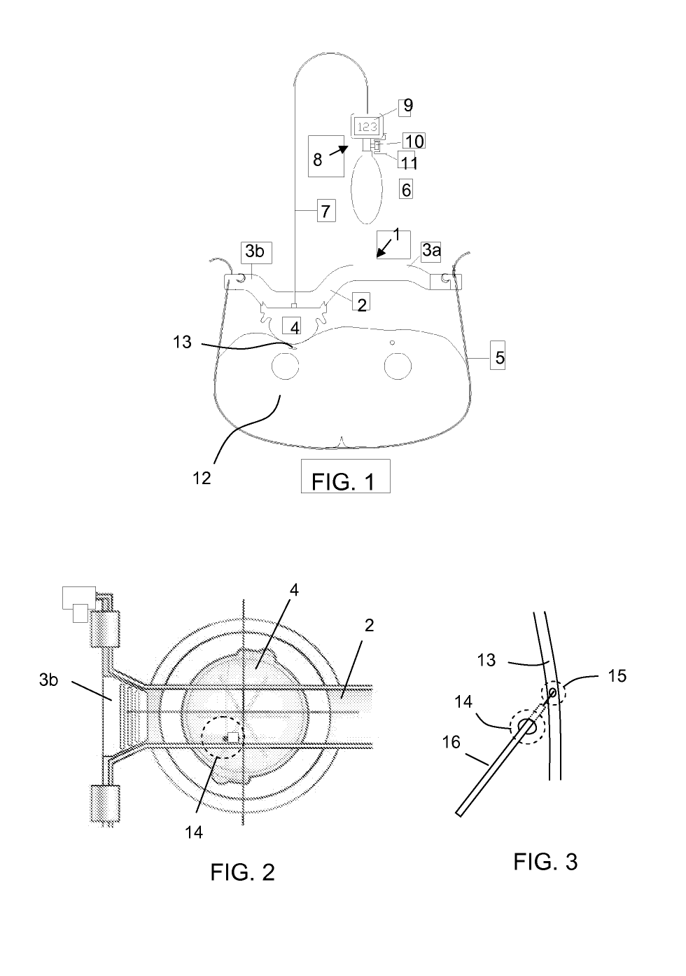

FIG. 1 illustrates a femoral compression device 1 as described in WO 2009/000665, and the present invention may be applied in connection with such device. The device 1 comprises a base plate 2 with two extensions 3a and 3b, a compression element 4, here in the shape of an inflatable and semi-spherical air cushion, a belt 5, a pump 6, an air connection 7, and an electric pressure gauge or manometer 8 with display 9. In use, compression element 4 is positioned over the femoral artery 13 of a patient 12, and the belt 5, which extends from the end of the first extension 3a, around the patient's body 12 and to the end of the second extension 3b, is tightened and secured by belt fasteners at the end of each extension. To apply pressure to the femoral artery 13, the inflatable semi-spherical air cushion 4 is inflated by the pump 6 to a certain pressure, which is measured by the manometer 8 and displayed on the display 9. The manometer 8 comprises further a vent knob 10, which is covered by a cap 11.

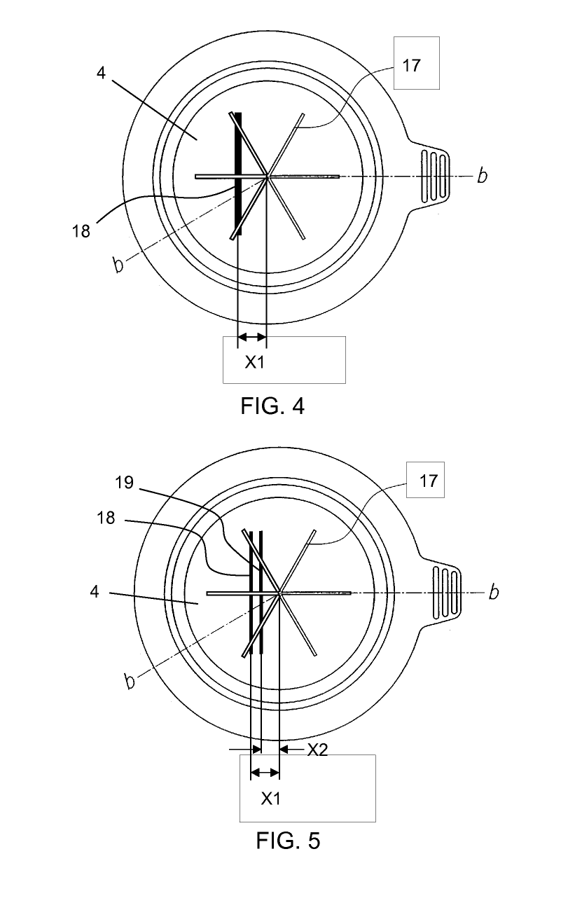

FIG. 2 illustrate the compression element 4, here in the shape of a semi-spherical air cushion, seen from above when positioned over a puncture site 15 (FIG. 3) in the femoral artery of a patient's body. The entrance site 14 through the skin for e.g. an introducer 16 is illustrated as a dot and is encircled by a dotted line. FIG. 3 is schematically illustrating the relationship between the entrance site 14 for e.g. an introducer 16 and the puncture site in a vessel 13. As noted from FIGS. 2 and 3, the entrance site 14 is situated inferior of and lateral of the puncture site 15. This applies to retrograde punctures.

It is very important to position the compression element 4 correctly over the puncture site 15, as otherwise the blood flow from the vessel 13 will not be stopped when the introducer 16 has been withdrawn. To guide a user in positioning the compression element 4, the present invention provides a compression system for compression against the puncture site 15 of a vessel 13 in a patient. The invention will now be described in conjunction with FIGS. 4 to 9, in which for simplicity only the compression element 4 adapted to apply a pressure against the puncture site 15 is shown, thus, the tightening unit 2, 3a, 3b, 5 adapted to extend around a part of, or the whole of, the patients body to fixate and to tighten the compression element against the puncture site 15, is here omitted. In the FIGS. 2, 4, 5, 7-9 the compression element 4 is illustrated from above when it is in its positioned state against a patient's body. The compression element 4 is here provided with reinforcement ribs or ridges 17 inside the cushion, here forming a star configuration.

According to the invention, the compression element 4 is provided with a placement mark 18, 19, 22, 23, 24, 25 indicating where the compression element 4 should be placed in relation to an entrance site 14 through the skin of the patient made to reach the puncture site 15, in order to apply pressure against the puncture site 15. By having such a mark on the compression element 4, the user is guided when positioning the compression element 4 and a correct pressure can be applied at the puncture site 15.

Preferably, the mark 18, 19, 22, 23, 24, 25 on the compression element 4 defines the entrance site 14 through the skin. Several embodiments are possible, and one example is illustrated in FIG. 4. A mark 18 is here provided lateral of a centre of the compression element 4, and in particular 1±0.5 centimetres lateral of the centre. The mark 18 is here illustrated as a longitudinal line 18, and the length and thickness of the longitudinal line 18 may differ according to various needs. The distance of the mark 18 lateral from the centre is denoted Xl. Advantageously the mark 18, 19, 22, 23, 24, 25 is adapted to fit over an introducer 16. The compression element 4 may then be positioned in relation to the introducer 16, thus, the compression element 4 is placed such that the longitudinal line 18 is located along the length of an introducer 16. According to one embodiment, the placement mark 18, 19 is provided in the direction of the vessel 13 to which the compression element 4 is applied. Thus, the longitudinal line or lines 18, 19 as illustrated in the figures, is or are then parallel to the direction of the vessel 13 when the compression element 4 is in use.

According to one embodiment as illustrated in FIG. 5, several marks 18, 19 are provided to define different placements of the compression element on a body with respect e.g. to age, length and/or weight of the patient. Age, length and weight are all factors which may affect the specific position of the puncture site 15 relative the entrance site 14 through the skin, and by having several marks on the compression element 4 reflecting these factors, these factors may be taken in account when positioning the compression element 4. In FIG. 5 two different distances X1 and X2 from a centre of the compression element to the marks 18, 19 are shown, but it is understood that there may be several more marks on the compression element 4 at different distances.

According to one embodiment, the placement mark 18, 19, 22, 23, 24, 25 on the compression element 4 is located on the surface 26 on the compression element 4 that is adapted to be pressed against the puncture site 15. According to another embodiment, the placement mark is located inside the compression element 4, and according to a further embodiment the placement mark is located on the surface on the compression element 4 opposite the surface 26 on the compression element 4 that is adapted to be pressed against the puncture site 15.

The placement mark 18, 19, 22, 23, 24, 25 on the compression element 4 is according to one embodiment a colour mark. The mark may be black or any other suitable colour visible for the user of the compression system. The compression element 4 is preferably made of a transparent material; such that the entrance site 14 is visible through the compression element 4, to facilitate the positioning of the placement mark 18, 19, 22, 23, 24, 25 above the entrance site 14.

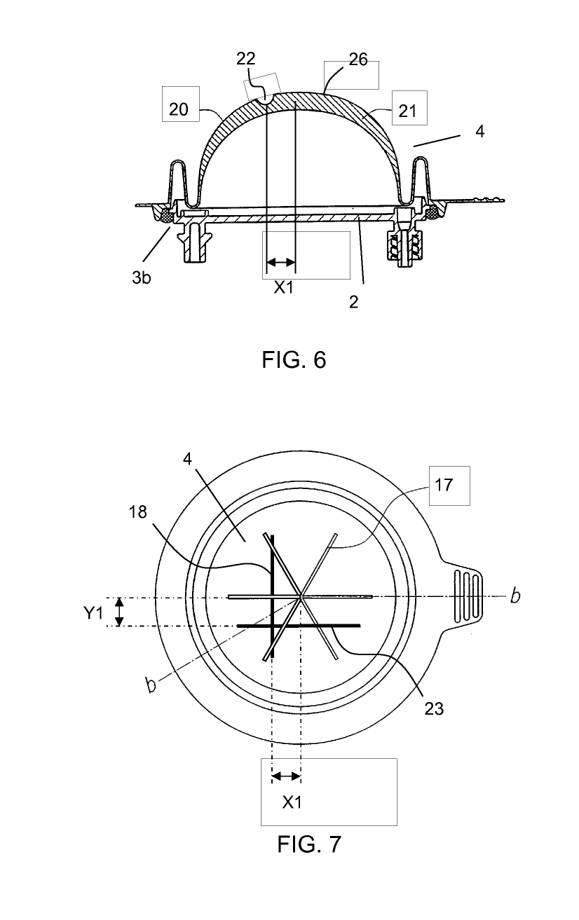

According to one embodiment, the placement mark 18, 19, 22, 23, 24, 25 on the compression element 4 is a depression 22. One example of such a depression 22 is shown in FIG. 6, located on the surface 26 of the compression element 4, and the distance of the depression 22 in relation to the centre of the compression element is denoted X1. The depression 22 may be longitudinal as explained in conjunction with FIGS. 4 and 5, and may be adapted to fit over an introducer 16. Withdrawal, or with other words, sheath pulling, of an introducer 16 may be facilitated as the introducer 16 may stay in place at the entrance site 14 when the compression element 4 is positioned, as the introducer 16 fits in the depression 22. The correct positioning of the compression element 4 is thus also guided by positioning the compression element 4 over the entrance site 14 such that the introducer 16 fits in the depression 22.

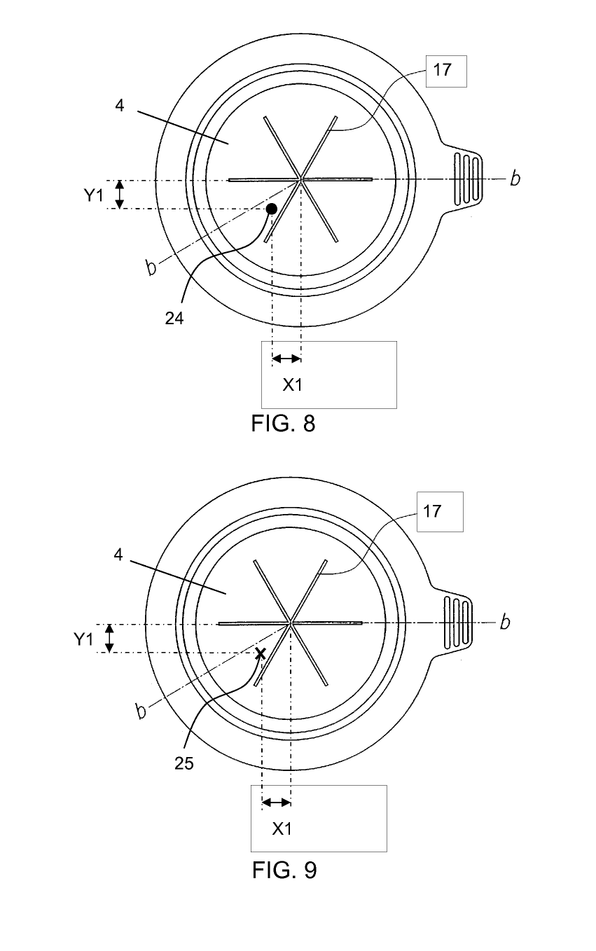

According to another embodiment as illustrated in FIG. 7, the compression element 4 comprises another placement mark 23 provided inferior of a centre of the compression element 4, and in particular 1±0.5 centimetres inferior of the centre. This distance of the placement mark 23 from the centre is denoted Y1 in the figure. In the figure, the two placement marks 18 and 23 shown as lines are intersecting at a point under which the entrance site 14 preferably is situated when the compression element 4 is used. This intersection may instead be denoted by a dot as illustrated in FIG. 8, by a cross as illustrated in FIG. 9 or by any other suitable denotation.

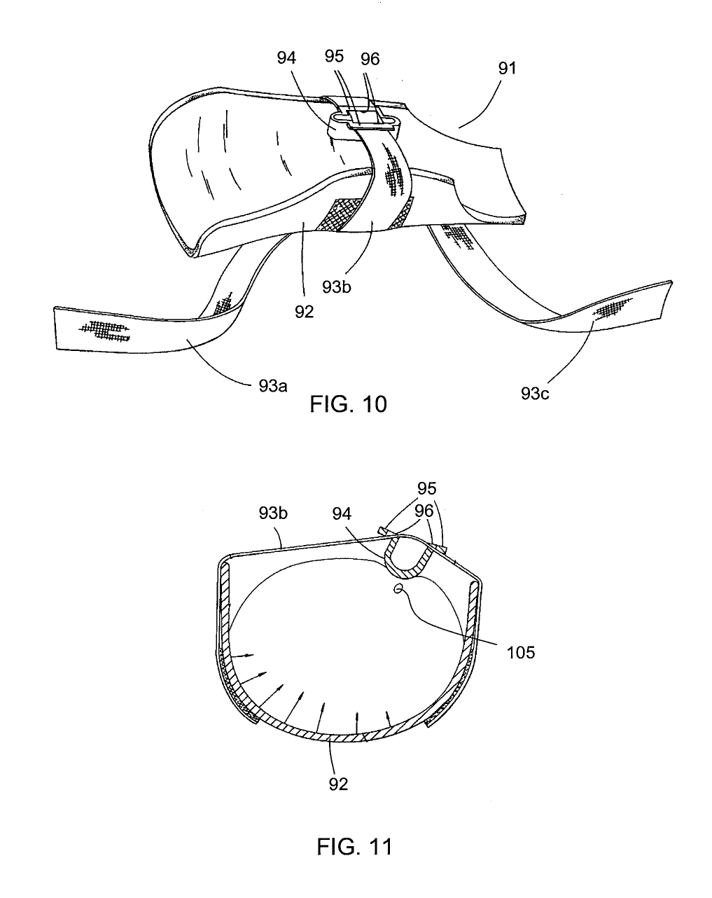

The present invention may also be used together with a device for compression of a radial artery. Such a device 91 is illustrated in FIGS. 10 and 11, and is known from WO 96/25110. The device 91 comprises in this embodiment a pressure distribution plate 92, three securing straps 93a, 93b, 93c provided with coupling means such as Velcro® fastenings or other suitable means. On the strap 93b there is provided a compression element 94, which here is elongated and extends in the direction of the artery 105 when in use. It has preferably a slightly rounded contact surface in order to smoothly bear on the skin. The compression element 94 has two flanges 96 each having a slot 97 through with the strap 93b is passed, to provide a sliding attachment of the compression element 94 to the strap 93b. The arrows in FIG. 11 from the plate 92 illustrate a major force component deviating from the perpendicular force from the compression element 94.

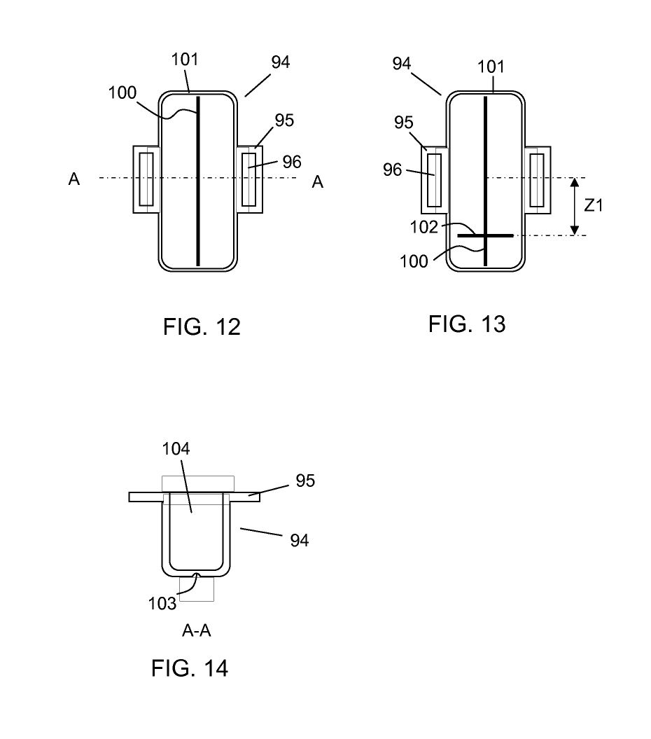

FIGS. 12 to 14 illustrate different embodiments of the invention, and will be described in the following. In the figures the tightening unit 92, 93a, 93b, 93c adapted to extend around a part of, or the whole of, the patients body to fixate and to tighten the compression element against the puncture site, has been omitted in order to simplify the figures. The compression element 94 is in these embodiments hollow, and the cavity is denoted 104 as can be seen from FIG. 14. In another embodiment not shown in the figures, the cavity 104 in the compression element is a through hole, and is adapted to receive an inflatable balloon which will exert a pressure on the puncture site when in use and inflated through the through hole. The placement mark which will be explained below will then be located on the inflatable balloon.

FIG. 12 illustrates a compression element 94 with a border 101, flanges 95 and slots 96. A placement mark 100 is provided on the compression element 94. The mark 100 may have any of the features as described above with reference to the compression element denoted 4, with the difference that the mark 100 is provided along a centre line of the compression element 94 in the direction of the vessel 105 on which the compression element 94 is intended to be applied when in use, as the entrance site through the skin to reach the radial vessel normally is right above the vessel. According to one embodiment as illustrated in the figures, the compression element 94 is elongated in the direction of a vessel to which it is applied.

In FIG. 13 another embodiment is illustrated comprising a second mark 102 provided inferior of the centre of the compression element 94, and which intersects with the previous mark 100. The intersection denotes the entrance site of an introducer, and the distance of the second mark 102 from a centre of the compression element 94 is denoted Z1. This distance is according to one embodiment 1±0.5 centimetres inferior of the centre of the compression element to make the centre of the compression element be located above the puncture site when the intersection is placed above entrance site. The intersection may instead of two intersecting lines 100, 102 be a dot or a cross positioned to denote the entrance site of the introducer.

According to an embodiment illustrated in FIG. 14, the mark 103 on the compression element 94 is a depression 103. The depression 103 may extend along said centre line of the compression element 94, in the surface intended to be pressed against the puncture site. The depression 103 may be adapted to fit over an introducer.

The compression element 94 is preferably made of a transparent material, such that the entrance site is visible through the compression element 94, to facilitate the positioning of the placement mark 100, 102, 103 above the entrance site.

The depression 22, 103 may according to one embodiment be provided on the compression element 4, 94 together with another kind of placement mark such as a second mark 23, 102 or a dot 24 or cross 25. The depression 22, 103 may according to a further embodiment be coloured for greater visibility.

As explained before, the present compression system is according to one embodiment adapted to be used for applying pressure on a patient's femoral artery. The compression system may according to another embodiment as explained above be adapted to be used for applying pressure on a patient's radial artery.

The present invention is not limited to the above-described preferred embodiments. Various alternatives, modifications and equivalents may be used. Therefore, the above embodiments should not be taken as limiting the scope of the invention, which is defined by the appending claims.

1-15. (canceled)

16. A compression system for compression against a puncture site of a vessel in a patient, comprising:
a compression element adapted to apply a pressure against said puncture site;
a tightening unit adapted to extend around a part of, or the whole of, the patient's body to fixate and to tighten the compression element against said puncture site;
wherein the compression element is provided with a placement mark indicating where the compression element should be placed in relation to an entrance site through the skin of the patient made to reach said puncture site, in order to direct pressure against said puncture site.

17. A compression system according to claim 16, wherein said placement mark is offset from an apex of the compression element.

18. A compression system according to claim 16, wherein said placement mark defines the entrance site through the skin.

19. A compression system according to claim 16, wherein said placement mark is located on the surface on the compression element that is adapted to be pressed against said skin.

20. A compression system according to claim 16, wherein said placement mark is a color mark.

21. A compression system according to claim 16, wherein said placement mark is a depression.

22. A compression system according to claim 16, wherein said placement mark is adapted to fit over an introducer.

23. A compression system according to claim 16, wherein said placement mark is provided in the direction of the vessel to which the compression element is applied.

24. A compression system according to claim 16, wherein said placement mark is provided 1±0.5 centimeters lateral of a center of said compression element.

25. A compression system according to claim 16, wherein the compression element comprises another placement mark provided 1±0.5 centimeters inferior of a center of said compression element.

26. A compression system according to claim 16, wherein said compression element is elongated in the direction of the vessel to which it is applied.

27. A compression system according to claim 26, wherein said placement mark is provided along a center line of the compression element.

28. A compression system according to claim 16, wherein the compression element is an inflatable cushion.

29. A compression system according to claim 16, wherein the compression element comprises several placement marks to define different placements of the compression element on a body based on age, length and/or weight of the patient.

30. A compression system according to claim 16 adapted to be used for applying pressure on the patient's femoral artery.

31. A compression system according to claim 16 adapted to be used for applying pressure on the patient's radial artery.

32. A compression system according to claim 16 wherein the placement mark is shaped to accommodate an introducer.

33. A method for applying compression against a puncture site of a vessel in a patient, comprising:
aligning a placement mark, positioned on a compressive element offset from an apex of the compressive element, with respect to an entrance site, through the skin of the patient, the entrance site being made to reach said puncture site; and
causing the compression element to direct pressure against said puncture site.

34. A method according to claim 33 wherein the placement mark is shaped to accommodate an introducer and the aligning comprises fitting together the placement mark and the introducer.

35. A method according to claim 33 wherein the compressive element is inflatable.

36. A method for applying compression against a puncture site of a vessel in a patient, comprising:
aligning a placement mark, positioned on a compressive element offset from a center point of the compressive element, with respect to an entrance site, through the skin of the patient, the entrance site being made to reach said puncture site; and
causing the compression element to direct pressure against said puncture site.
